(19) United States
(12) Patent Application Publication
(10) Pub. No.: US 2020/0077937 A1
(43) Pub. Date: Mar. 12, 2020

(54) SYSTEM AND METHOD FOR COMPREHENSIVE MULTISENSORY SCREENING

(71) Applicant: IVISION TECHNOLOGIES, LLC, Des Plaines, IL (US)

(72) Inventors: Stuart Paul Richer, Highland Park, IL (US); Robert Endo, Northbrook, IL (US); Douglas K. Oshana, Lincolnwood, IL (US)

(73) Assignee: IVISION TECHNOLOGIES, LLC, Des Plaines, IL (US)

(21) Appl. No.: 16/563,551

(22) Filed: Sep. 6, 2019

Related U.S. Application Data
(60) Provisional application No. 62/728,039, filed on Sep. 6, 2018.

(51) Int. Cl. A61B 5/00; G16H 10/60; G06N 20/00; A61B 5/117

(52) U.S. Cl. CPC A61B 5/4005; A61B 5/7475; A61B 5/117; G06N 20/00; A61B 5/6814; G16H 10/60

(57) ABSTRACT

A system for comprehensive multisensory vision, hearing, and cognitive screening includes a headwear test unit comprising a configurable video, audio, and hand-gesture capable testing device. The system further includes an identification, anonymization and security module, a patient interface, an operator interface, a communication module, and a unit charging and calibration module and/or station. An adaptive real-time compiler compiles sequences of optometric hearing and cognition tests. A cloud-based web service module is configured for storing encrypted personal optometric information. A machine learning module is operatively connected to the cloud-based web service module.

This application is a non-provisional of and claims the benefit of U.S. Provisional Patent Application No. 62/728,039 filed Sep. 6, 2018, the entire disclosure of which is incorporated herein by reference. The disclosures of U.S. Provisional Patent Application No. 62/728,044 filed Sep. 6, 2018 and U.S. Provisional Patent Application No. 62/728,037 filed Sep. 6, 2018 are also incorporated herein by reference in their entirety.

This application includes material which is subject to copyright protection. The copyright owner has no objection to the facsimile reproduction by anyone of the patent disclosure, as it appears in the Patent and Trademark Office files or records, but otherwise reserves all copyright rights whatsoever.

### FIELD

The present invention relates in general to the field of screening devices and methods for vision, auditory and cognitive screening.

### SUMMARY

In general, example embodiments of the present invention provide a unique and innovative system and method for vision, hearing, cognition and proprioception testing as the key features of the system. Proposed system and method facilitate greater efficiency and throughput of patient flow, and to enable contemporary achievements in video and communication technologies, providing customer self-paced vision test capabilities, while minimizing time and interactions with medical personnel.

### BRIEF DESCRIPTION OF THE DRAWINGS

The foregoing and other objects, features, and advantages of the invention will be apparent from the following more particular description of preferred embodiments as illustrated in the accompanying drawings, in which reference characters refer to the same parts throughout the various views. The drawings are not necessarily to scale, emphasis instead being placed upon illustrating principles of the invention.

FIG. 1 shows a graphical view illustrating a process in accordance with an embodiment of the invention.

FIG. 2 shows a state diagram in accordance with an embodiment of the invention.

FIG. 3 shows a block diagram illustrating a configuration of software modules in accordance with an embodiment of the invention.

FIG. 4A shows a flow diagram illustrating testing of a patient's sensory acuity.

FIG. 4B shows a flow diagram illustrating a test sequence in accordance with an embodiment of the invention.

FIG. 5 shows a flowchart illustrating a method for testing in accordance with an embodiment of the invention.

### DETAILED DESCRIPTION

Reference will now be made in detail to the preferred embodiments of the present invention, examples of which are illustrated in the accompanying drawings. The following description and drawings are illustrative and are not to be construed as limiting. Numerous specific details are described to provide a thorough understanding. However, in certain instances, well-known or conventional details are not described in order to avoid obscuring the description. References to one or an embodiment in the present disclosure are not necessarily references to the same embodiment; and, such references mean at least one.

Reference in this specification to “an embodiment” or “the embodiment” means that a particular feature, structure, or characteristic described in connection with the embodiment is included in at least an embodiment of the disclosure. The appearances of the phrase “in an embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

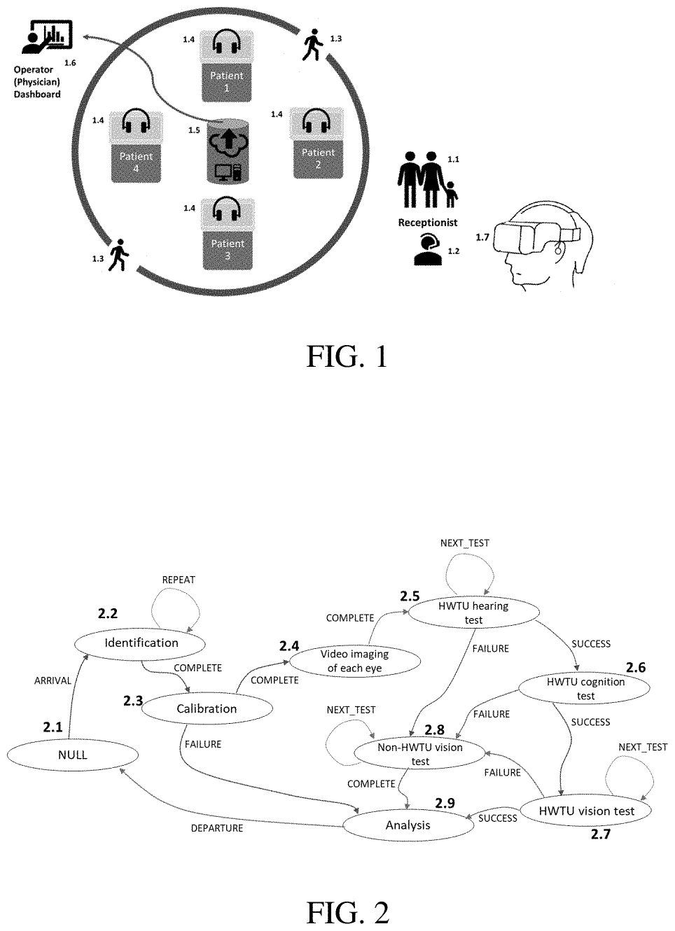

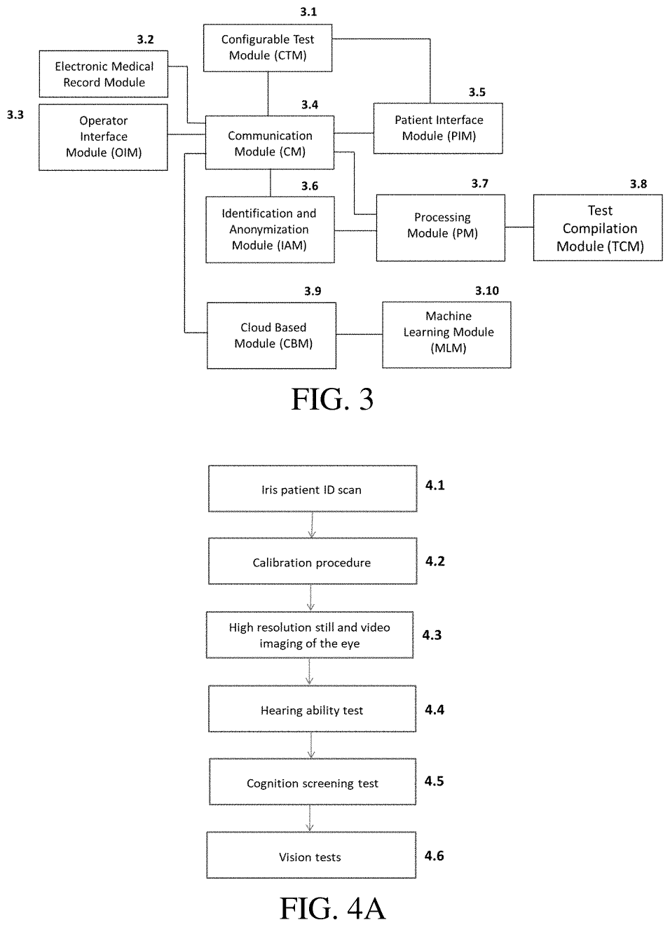

According to an embodiment shown in FIG. 1, a patient (1.1) checks in with receptionist (1.2) and then sits down in one of patient chairs (1.4) picks up test device attached to a central service column. The test device turns on and off by lifting the head ware unit (1.7) from the cradle. According to some embodiments a circular or flat sound proof partition has two or more entrances/exits (1.3). Approximately ninety degree of arc for each of the patients is provided for him/her to use hand gestures but more or less space is also possible. The central “service column” (1.5) has all the service hardware that comprises at least a processor, charging station, lift cradles, camera, speakers, test devices (a headwear test unit, HWTU), wired and wireless communication means. The information collected from the test devices is communicated to the operator dashboard (1.6), which tracks status and progress off all patients under test including providing help in audio and/or visual format(s) if patient gets stuck or confused as well as the status of patients waiting in the queue.

The use of head ware test unit, HWTU, has at least three advantages: (i) employment of calibrated lighting during vision tests, (ii) implementation of various methods that that minimizes cheating during screening and testing, and (iii) unlike standard vision testing, eliminates patient's moving forward in his/her chair to gain an advantage in increasing chances of correct answers.

State machine of FIG. 2 is an example embodiment that may be embodied by or associated with any of a variety of vision test sequences that include or are otherwise associated with a vision test system and method.

According to other embodiments, a HWTU calibration procedure may be executed before a patient enters the Identification state, especially if HWTU has any moving parts for alignment.

According to some embodiments, a patient may at any moment of time during the test procedure be in one of the seven logical states. When patient puts on HWTU, he/she enters the Identification state (2.2) from the NULL state (2.1) where the identification procedure is executed. A patient then enters the Calibration state (2.3), where the test unit is calibrated per patient specific features (Note that calibration and identification states order can be reversed). After which he/she enters the Imaging state (2.4) where high resolution still and video imaging of the eye and adnexa is performed. When all imaging procedures are completed a user enters the HWTU hearing test state (2.5). If the hearing test fails then patient enters the standard non-HWTU vision test (2.8). Otherwise the HWTU cognition test state is entered followed by the HWTU vision test state in case of success or non-HWTU vision test (2.8) otherwise. A patient enters the Analysis state 2.9 from Calibration, non-HWTU vision test and HWTU vision test states. Patient returns to the NULL state upon all procedures in Analysis state are completed.

According to an example embodiment, the vision system comprises several modules. All communication among modules is performed and controlled by the Communication Module (CM). The CM exchanges information with the Configurable Test Module (CTM), a.k.a. test device or a head ware test unit (HWTU), the Patient Interface Module (PIM) that is connected to CTM, Identification and Anonymization Module (IAM) which performs patient identification procedure as well anonymizes said information with the aim to be compliant with the laws and regulations. In addition, the CM communicates with the Operator Interface Module (OIM) (see FIG. 1, 1.6), Electronic Medical Record Module as well as the Cloud Based Module which is used for secure storing information as well as the post processing performed by the Machine Learning Module (MLM). Finally, the Processing Module (PM) is connected to the Test Compilation Module (TCM), IAM and CM.

The TCM is used, for example, for modifying in real-time a sequence of vision tests based on the pre-test execution results.

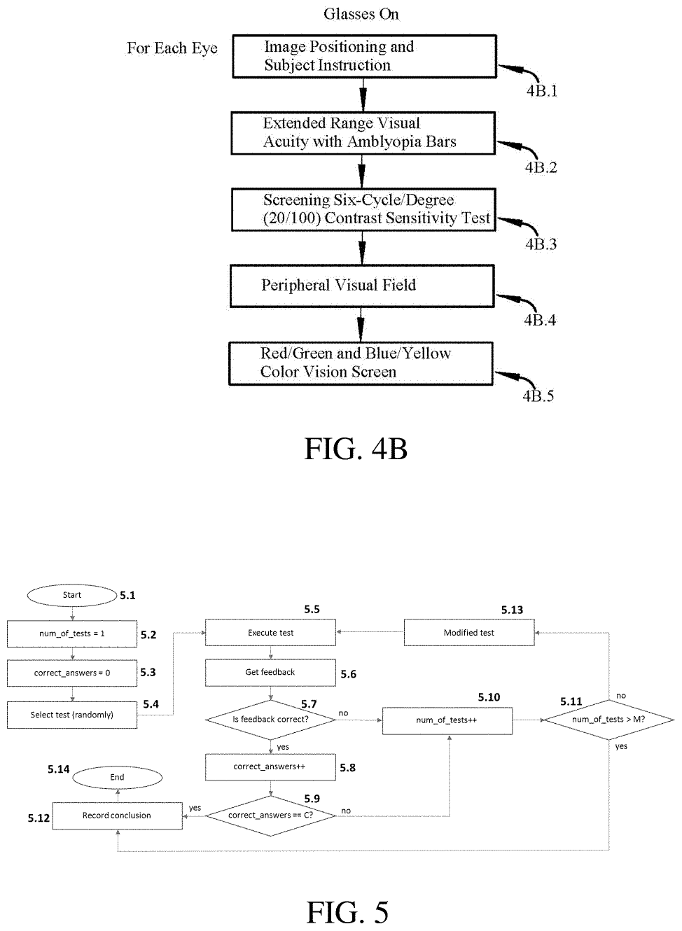

According to some embodiments, the method for multisensory screening comprises six sequentially executed procedures presented in FIG. 4A.

In yet another embodiment testing a patient's sensory acuity (proprioception assessment) can be added to the hearing, cognition and vision screening. Also, order of calibration and ID scan can be reversed.

The patient identification 4.1 that can, for example, be based on the iris scan and/or other methods that support unique identification is intended to generate a reliable and HIPPA compliant patient ID. Identification and confirmation of the patient can be one by scanning the iris using HWTU cameras or any other methods using computer vision, audio, password or combination of these methods.

In an embodiment, the calibration procedure 4.2 is at least the following:
a. The HWTU displays luminance must be measured in candela per square meter and adjusted to one of three pre-defined levels of scotopic, mesopic and photopic visions.
b. The position of images presented to the patient in the HWTU must be adjusted depending on the line of view.
c. The ocular fixation calibration must be performed to determine foveal gaze in each eye.
d. Auto-Calibration of each screen for color (chromaticity coordinates), saturation and luminance for accurate and precise color vision testing and gauging whether screens are deteriorating.
e. Remote access to HWTU calibration data.

As part of the 4.2 procedure the system needs to make sure that no light is leaking through the sides of the head unit during calibration and tests.

In addition to image calibration the system will have capability for automated audio calibration.

The high resolution still and video imaging of the eye procedure 4.3 is performed by HWTU cameras and includes:
Measurement of blink rate and incomplete blinks
Automated measurement of pupil size/afferent defect
Measurement of inter-pupil distance
Measurement of the pupil size in light with the highest diffuse HWTU display luminance available
Measurement of the pupil size and reaction time in dark with the HWTU display luminance low or off
Measurement of pursuits and saccade eye movements
Measurement of glare response using bright lights from the LED module
Marcus Gunn test comprising:
   Tests Cranial Nerve II
   Measurement of direct response to light
   Measurement of consensual response to light
   Measurement of afferent pupillary defect

Note that the procedures 4.1, 4.2 and 4.3 must be performed without glasses and no patient response is required, albeit a patient must follow instructions.

In some embodiments the HWTU cameras are used to capture opacification of the human lens i.e. qualitative image and quantitative density of the cataract of the human lens with each eye after dilation of the eyes by the doctor.

The aim of the hearing ability test 4.4 is twofold:

a. Screening patient's hearing function as this is the major sensory system for learning for 75% of the population, and b. Adjust the sound level of the HWTU headphones to make sure that the audio screening instructions and question are clearly understood

In some embodiments said hearing test comprises additional screening including but not limited to the high and low frequency tests.

The cognition screening test 4.5 implements in HWTU basic visual and auditory stimuli requirements for cognition assessment according to the methodology from “Sensory dominance and multisensory integration as screening tools in aging” Micah M. Murray, Alison F. Eardley, Trudi Edginton6, Rebecca Oyekan5, Emily Smyth5 & Pawel J. Matuszl, NATURE-SCIENTIFIC Reports I (2018) 8:8901 I DOI:10.1038/s41598-018-27288-2.

In yet another embodiment the calibration procedure is accomplished in non-ware cradle mode instead of HWTU mode.

Finally, comprehensive vision screening tests 4.6 are executed in a predefined order, but in another embodiment the order can be changed and augmented with other tests. Some embodiments may include a test that uses a spectrophotometer to measure the spectral characteristics of the eyeglasses the patient is wearing as well as the peripheral vision testing using HWTU mounted LED modules. The spectrophotometer sensor can be part of HWTU or the spectrophotometer can be part of an external fixture. The corresponding test sequence is shown in the FIG. 4B.

It should be noted that the sequence of tests presented in the FIG. 4B consists of 4 monocular and one binocular test.

The full list of vision tests is present in the TABLE 1:

TABLE 1

| | Test | Patient Response | Comments |
|---|---|---|---|
| 1 | Screening Contrast test | alpha | show one row at a time |
| 2 | Landolt C | direction | |
| 3 | Letter range | alpha | Acuity test |
| 4 | Amblyopia test | alpha | Acuity test |
| 5 | Spatial vision | alpha | |
| 6 | Hand motion | binary + direction | |
| 7 | Confrontational visual field | alpha | |
| 8 | Red desaturation | sliding bar | |
| 9 | R/G and B/Y color vision | numeric | |
| 10 | Full Contrast sensitivity | alpha | |
| | function | | |
| 11 | Mesopic motion sensitivity | binary + direction | |
| 12 | High contrast visual activity | sliding bar | |
| 13 | Glare disability | alpha | |
| 14 | Useful field of view | object + direction | |
| 15 | Efficient contrast sensitivity | alpha | one row at a time for 4 remaining |
| | | | spatial frequencies |
| 16 | Dark field/IR illumination | none | Evaluate cataract |
| 17 | Worth 4 dot Suppression | numeric | |
| 18 | Vertical Phoria | numeric | |
| 19 | Horizontal Phoria | numeric | Same presentation as 18 |
| 20 | Fixation disparity | numeric | |
| 21 | Randot stereopsis | shape | |
| 22 | Amsler grid | binary + alpha | |
| 23 | Glare recovery | direction | |
| 24 | Macular Pigment Optical | numeric | For each eye |
| | Density Test | | |

The vision testing system shall increase the probability of patient test output that reflects real vision condition. With this aim, each test that requires patient feedback will be repeated predefined number of times M and the patient's feedback will be recorded according to the following rule: The answer is concluded as a correct one if and only if a predefined number of answers C out of M test repetitions are correct answers. Otherwise the answer is concluded as an incorrect one. The flowchart depicting the proposed method is shown in FIG. 5.

The present invention is described above with reference to block diagrams and operational illustrations of methods and devices for comprehensive multisensory screening. It is understood that each block of the block diagrams or operational illustrations, and combinations of blocks in the block diagrams or operational illustrations, may be implemented by means of analog or digital hardware and computer program instructions. These computer program instructions may be stored on computer-readable media and provided to a processor of a general-purpose computer, special purpose computer, ASIC, or other programmable data processing apparatus, such that the instructions, which execute via the processor of the computer or other programmable data processing apparatus, implements the functions/acts specified in the block diagrams or operational block or blocks. In some alternate implementations, the functions/acts noted in the blocks may occur out of the order noted in the operational illustrations. For example, two blocks shown in succession may in fact be executed substantially concurrently or the blocks may sometimes be executed in the reverse order, depending upon the functionality/acts involved.

At least some aspects disclosed can be embodied, at least in part, in software. That is, the techniques may be carried out in a special-purpose or general-purpose computer system or other data processing system in response to its processor, such as a microprocessor, executing sequences of instructions contained in a memory, such as ROM, volatile RAM, non-volatile memory, cache or a remote storage device. Functions expressed in the claims may be performed by a processor in combination with memory storing code and should not be interpreted as means-plus-function limitations.

Routines executed to implement the embodiments may be implemented as part of an operating system, firmware, ROM, middleware, service delivery platform, SDK (Software Development Kit) component, web services, or other specific application, component, program, object, module or sequence of instructions referred to as “computer programs.” Invocation interfaces to these routines can be exposed to a software development community as an API (Application Programming Interface). The computer programs typically comprise one or more instructions set at various times in various memory and storage devices in a computer, and that, when read and executed by one or more processors in a computer, cause the computer to perform operations necessary to execute elements involving the various aspects.

A machine-readable medium can be used to store software and data which when executed by a data processing system causes the system to perform various methods. The executable software and data may be stored in various places including for example ROM, volatile RAM, non-volatile memory and/or cache. Portions of this software and/or data may be stored in any one of these storage devices. Further, the data and instructions can be obtained from centralized servers or peer-to-peer networks. Different portions of the data and instructions can be obtained from different centralized servers and/or peer-to-peer networks at different times and in different communication sessions or in a same communication session. The data and instructions can be obtained in entirety prior to the execution of the applications. Alternatively, portions of the data and instructions can be obtained dynamically, just in time, when needed for execution. Thus, it is not required that the data and instructions be on a machine-readable medium in entirety at a particular instance of time.

Examples of computer-readable media include but are not limited to recordable and non-recordable type media such as volatile and non-volatile memory devices, read only memory (ROM), random access memory (RAM), flash memory devices, removable disks, magnetic disk storage media, optical storage media (e.g., Compact Disk Read-Only Memory (CD ROMS), Digital Versatile Disks (DVDs), etc.), among others.

In general, a machine-readable medium includes any mechanism that provides (e.g., stores) information in a form accessible by a machine (e.g., a computer, network device, personal digital assistant, manufacturing tool, any device with a set of one or more processors, etc.).

In various embodiments, hardwired circuitry may be used in combination with software instructions to implement the techniques. Thus, the techniques are neither limited to any specific combination of hardware circuitry and software nor to any particular source for the instructions executed by the data processing system.

As used herein, and especially within the claims, ordinal terms such as first and second are not intended, in and of themselves, to imply sequence, time or uniqueness, but rather are used to distinguish one claimed construct from another. In some uses where the context dictates, these terms may imply that the first and second are unique. For example, where an event occurs at a first time, and another event occurs at a second time, there is no intended implication that the first time occurs before the second time. However, where the further limitation that the second time is after the first time is presented in the claim, the context would require reading the first time and the second time to be unique times. Similarly, where the context so dictates or permits, ordinal terms are intended to be broadly construed so that the two identified claim constructs can be of the same characteristic or of different characteristic.

While some embodiments can be implemented in fully functioning computers and computer systems, various embodiments are capable of being distributed as a computing product in a variety of forms and are capable of being applied regardless of the particular type of machine or computer-readable media used to actually effect the distribution.

The above embodiments and preferences are illustrative of the present invention. It is neither necessary, nor intended for this patent to outline or define every possible combination or embodiment. The inventors have disclosed sufficient information to permit one skilled in the art to practice at least one embodiment of the invention. The above description and drawings are merely illustrative of the present invention and that changes in components, structure and procedure are possible without departing from the scope of the present invention as defined in the following claims. For example, elements and/or steps described above and/or in the following claims in a particular order may be practiced in a different order without departing from the invention. Thus, while the invention has been particularly shown and described with reference to embodiments thereof, it will be understood by those skilled in the art that various changes in form and details may be made therein without departing from the spirit and scope of the invention.

What is claimed is:

1. A system for comprehensive multisensory vision, hearing, and cognitive screening, the system comprising:
a headwear test unit comprising a configurable video, audio, and hand-gesture capable testing device;
an identification, anonymization and security module;
a patient interface;
an operator interface;
a communication module configured to transmit and receive information via a wired or wireless interface;
a unit charging and calibration modules and/or station;
an adaptive real-time compiler of sequences of optometric hearing and cognition tests;
a cloud-based web service module configured for storing encrypted personal optometric information;
an electronic medical record module; and,
a machine learning module operatively connected to said cloud-based web service module.

2. The system for comprehensive multisensory testing in accordance with claim 1, wherein the system comprises multiple stations and is configured to handle multiple patients simultaneously.

3. The system for comprehensive multisensory testing in accordance with claim 2, wherein each station is capable of screening vision, hearing, and/or cognition abilities.

4. The system for comprehensive multisensory testing in accordance with claim 2, wherein each station is configured to capture opacification of a human lens with each eye after dilation of the eyes by a doctor.

5. The system for comprehensive multisensory testing in accordance with claim 1, wherein said patient interface is configured to handle multiple patient head sizes, anatomies with and without glasses, and a wide range of vision acuity.

6. The system for comprehensive multisensory testing in accordance with claim 1, wherein said patient interface is configured to create alphanumeric, audio, directional static and dynamic requests to a patient.

7. The system for comprehensive multisensory testing in accordance with claim 1, wherein said patient interface is configured for colleting customer input comprising one or more of:
voice, gesture, blinks, eye movement, head movement, foot taps, keyboard input, and mouse input.

8. The system for comprehensive multisensory testing in accordance with claim 1, wherein said operator interface is configured to schedule and track progress of each patient as well as provide assistance if a patient becomes stuck or confused.

9. The system for comprehensive multisensory testing in accordance with claim 1, wherein said Machine Learning Module is configured to analyze and evaluate data across a plurality of patients, ranking and grading patients across known population and identifying acuities and inconsistencies across tests.

10. A comprehensive multisensory method for vision, binaural screening of hearing with automated audio decibel adjustment, and cognitive screening, the method comprising the steps of:
performing an iris patient ID scan;
performing a calibration procedure;
performing high resolution still and video imaging;
performing a hearing ability test and audio adjustment;
performing a cognition screening test; and,
performing a vision test.

11. A vision test system, comprising:
a patient identification iris scanning station;
a charging station;
a calibration station;
at least one test station having:
a headwear device;
a transceiver communicatively coupled with said headwear device via a wired or wireless link;
a first set of monocular optometry tests;
a second set of optometry tests;
a real-time optometry test sequence generator configured to generate a test sequence based upon age of a patient and response modality (patient's ability to respond with audio/visual) results from an initial test, the test sequence generator compiling a third set of sequential tests based on the age of the patient; and,
a spectrophotometer configured to measure spectral characteristics of eyeglasses.
